(19) United States
(12) Patent Application Publication
(10) Pub. No.: US 2017/0021200 A1
(43) Pub. Date: Jan. 26, 2017

(54) LOW FREQUENCY ULTRASONIC VIBRATION ASSISTED BLEEDING-LESS SURGICAL TOOL

(71) Applicants: Joanna Le Xi LI, La Jolla, CA (US); Qian XIA, Singapore (SG); Xiaoping LI, Singapore (SG)

(72) Inventors: Joanna Le Xi LI, La Jolla, CA (US); Qian XIA, Singapore (SG); Xiaoping LI, Singapore (SG)

(21) Appl. No.: 15/216,356

(22) Filed: Jul. 21, 2016

Related U.S. Application Data
(60) Provisional application No. 62/195,277, filed on Jul. 21, 2015.

(51) Int. Cl. A61N 7/02; A61B 17/32

(52) U.S. Cl. CPC A61N 7/02; A61B 17/320068; A61B 2018/00398

(57) ABSTRACT

The present invention is a method for a bleedingless surgical tool that induces cavitation in blood vessel, tissue, and/or body fluid to facilitate platelet plug formation and induces cavitation together with a temperature rise to facilitate fibrin formation, inducing and enhancing blood clotting such that bleeding does not occur while cutting is in progress. The present invention is further a bleedingless surgical tool comprising a cutting portion of the surgical tool and an ultrasonic vibration generating portion forming by an ultrasonic alternating power source and an ultrasonic vibration transducer, which during cutting of live tissues and blood vessels induces cavitation in the blood vessel, tissue, and/or body fluid to facilitate platelet plug formation and induces cavitation and temperature rise to facilitate fibrin formation, inducing and enhancing blood clotting such that bleeding does not occur while the cutting is in progress.

### RELATED APPLICATIONS

The present invention corresponds to the U.S. Provisional Patent Application 62/195,277, which has the title: “LOW FREQUENCY ULTRASONIC VIBRATION ASSISTED BLEEDING-LESS SURGICAL TOOL”.

### FIELD OF THE INVENTION

The present invention is related to methods and apparatus for bleeding-less surgical tools.

### BACKGROUND OF THE INVENTION

It has been reported that 29.9% of surgeries surveyed experienced complications due to blood-loss and other complications due to bleeding. There are many cases where uncontrollable bleeding after surgeries has led to death, even among simple surgical procedures. Bleeding during surgery poses a grave threat to the safety of many patients, resulting in expensive aspiratory devices and the need for blood transfusions during the surgery. It is exigent to create surgical tools, which can staunch bleeding in surgery while cutting the tissues and blood vessels. The most prevalent solution to this problem is through cauterization—whether it be laser, high frequency ultrasonic vibration, or electrical cauterization. However, these methods can lead to permanent tissue damage as well as infections since they work through thermal damaging of the tissue cells. Furthermore, a period of time is required for the damaged cells to recover from cauterization. The present invention is a low frequency ultrasonic vibration assisted surgical tool, which cuts tissues and blood vessels while staunching bleeding effectively by inducing and enhancing blood clotting, without thermal cauterization/damage to surrounding tissues cells.

### FEATURES OF THE INVENTION

The first feature of the present invention is to have a bleeding-less surgical tool that induces cavitation in the blood vessel, body fluids, and/or tissue under cutting for blood clotting.

The second feature of the present invention is to have a bleeding-less surgical tool with ultrasonic vibration, in which while the tool is in cutting the tool ultrasonic vibration induces cavitation in the blood vessel, body fluids, and/or tissue under cutting and enhances blood clotting.

The third feature of the present invention is to have a bleeding-less surgical tool with low frequency ultrasonic vibration for inducing and enhancing blood clotting, in which the ultrasonic vibration frequency is so low that there will be no thermal damages to the tissues surrounding the tool.

The forth feature of the present invention is to have a wire/cable connecting the ultrasonic vibration transducer and ultrasonic alternating power source in the bleeding-less surgical tool with ultrasonic vibration.

The fifth feature of the present invention is to have a wireless connection connecting the ultrasonic vibration transducer and ultrasonic alternating power source in the bleeding-less surgical tool with ultrasonic vibration.

### BRIEF DESCRIPTION OF THE DRAWINGS

FIG. 1 shows a method of the present invention—a surgical tool with low frequency ultrasonic vibration facilitating platelet plug formation and fibrin formation and thus inducing and enhance blood clots at the cut tissues surrounding the tool, leading to blooding-less surgical operation.

FIG. 2 shows one embodiment of the present invention, a low frequency ultrasonic vibration assisted surgical tool consisting of a surgical knife connected to an ultrasonic vibration transducer driven by a low frequency ultrasonic alternating power source, having the knife vibrating along the axial direction of the knife at the ultrasonic frequency.

FIG. 3 shows another embodiment of the present invention, a low frequency ultrasonic vibration assisted surgical tool consisting of a surgical knife connected to an ultrasonic vibration transducer driven by a low frequency ultrasonic alternating power source, in which the ultrasonic vibration transducer is connected to the low frequency ultrasonic alternating power source with a wire connection.

FIG. 4 shows another embodiment of the present invention, a low frequency ultrasonic vibration assisted surgical tool consisting of a surgical knife connected to an ultrasonic vibration transducer driven by a low frequency ultrasonic alternating power source, in which the ultrasonic vibration transducer is connected to the low frequency ultrasonic alternating power source with a wireless connection.

### DESCRIPTION OF THE INVENTION

The present invention is a bleeding-less surgical tool, in which the tool induces cavitation in blood vessel, tissue, and/or body fluid with low frequency ultrasonic vibration so that as the tool cuts live tissues and blood vessels it also induces and enhances blood clotting such that bleeding does not occur while the cutting is in progress.

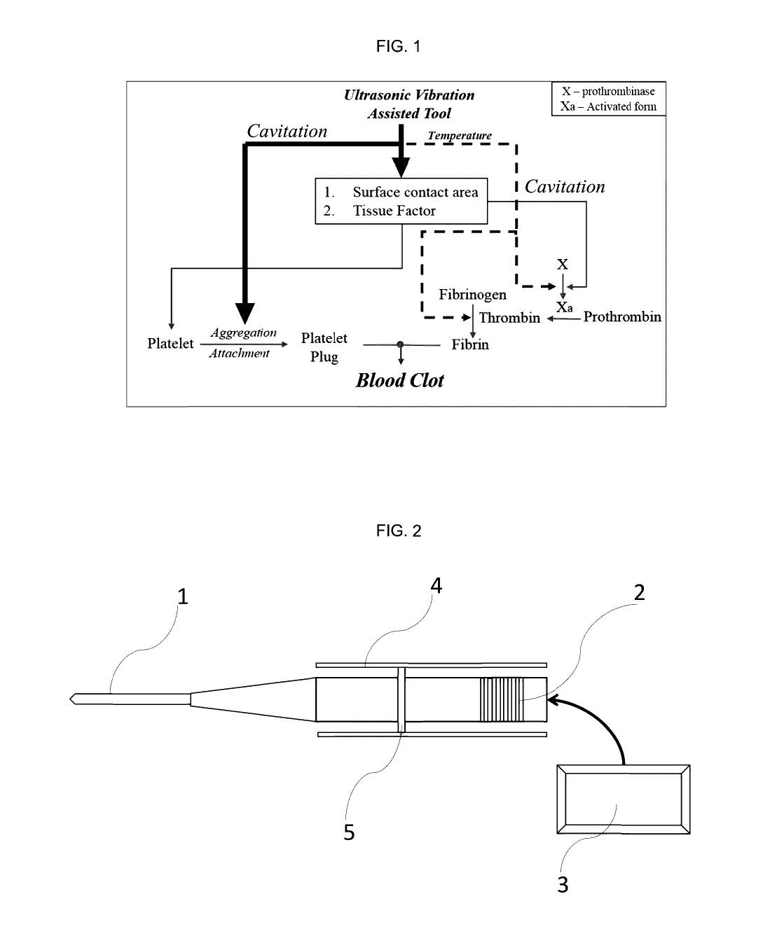

FIG. 1 shows the present invention that as the surgical tool with low frequency ultrasonic vibration cuts a live blood vessel the tool ultrasonic vibration acts on inducing blood clotting in two ways simultaneously: 1) facilitating platelet plug formation by greatly increasing the surface contact areas and tissue factors through cavitation induced in blood vessel, tissue, and/or body fluid such that platelets are greatly activated and aggregated by the excessive collagen exposed by cavitation-caused tissue rupture and jetting effect resulting from the violent collapse of a bubble during cavitation which can rip apart platelets and release platelet contents 2) facilitating the fibrin formation by increasing the local blood temperature and inducing cavitation in blood vessel, tissue, and/or body fluid so that prothrombin is greatly activated and fibrin repair is stimulated, and thus increasing thrombin formation, such that fibrinogen is greatly transformed into fibrin. The combining of facilitations of platelet formation and fibrin formation greatly enhances blood clotting. And yet, the ultrasonic vibration is in a low frequency range so that local tissue cell damages caused by burning at the tool-tissue interface is avoided.

FIG. 2 shows one of the ways of using cavitation-inducing bleeding-less surgical tool is by using a low ultrasonic frequency vibration driven bleeding-less surgical tool of the present invention which may be formed by a surgical knife 1 connected an ultrasonic vibration transducer 2, which can be a piezoelectric transducer, a capacitive transducer or a magnetostriction transducer, driven by a low frequency ultrasonic alternating power source 3, having a holder 4 mounted onto the ultrasonic vibration wave node 5 of the transducer 2. During a surgical operation the low ultrasonic frequency vibration assisted bleeding-less surgical knife is held to cut the tissues/blood vessels while the knife 1 vibrates along the axial direction at a low ultrasonic frequency to induce and enhance blood clots in tissues/blood vessels surrounding the knife, as shown in FIG. 1, making bleeding-less surgical operation.

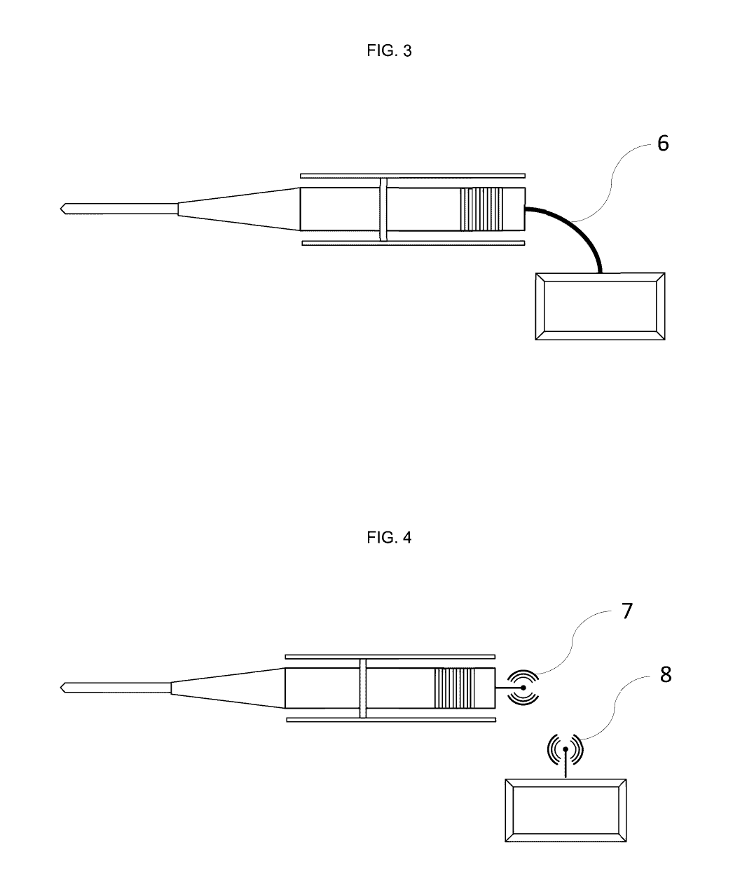

FIG. 3 shows that in FIG. 2 the ultrasonic vibration transducer 2 and the low frequency ultrasonic alternating power source 3 are connected with a wire connection, in which a wire/cable 6 is connected to ultrasonic vibration transducer 2 and the low frequency ultrasonic alternating power source 3.

FIG. 4 shows that in FIG. 2 the ultrasonic vibration transducer 2 and the low frequency ultrasonic alternating power source 3 are connected with a wireless connection, in which the low frequency ultrasonic alternating power source 3 has an antenna 8 transmitting the alternating power from the power source 3 to the ultrasonic vibration transducer 2, and the ultrasonic vibration transducer 2 has another antenna 7 receiving the alternating power transmitted by antenna 8.

1. Method and apparatus for a bleedingless surgical tool that induces cavitation in blood vessel, tissue, and/or body fluid to facilitate platelet plug formation and induces cavitation together with a temperature rise to facilitate fibrin formation, inducing and enhancing blood clotting such that bleeding does not occur while cutting is in progress

2. In claim 1, the bleedingless surgical tool can comprise a cutting portion of the surgical tool and an ultrasonic vibration generating portion forming by an ultrasonic alternating power source and an ultrasonic vibration transducer, which during cutting of live tissues and blood vessels induces cavitation in the blood vessel, tissue, and/or body fluid, inducing and enhancing blood clotting such that bleeding does not occur while the cutting is in progress.

3. In claim 2 the cutting portion of the bleedingless surgical tool can be the same cutting portion as per normal of a surgical tool.

4. In claim 2 the direction of the ultrasonic vibration can be in any direction, such as in the axial direction of the surgical tool.

5. In claim 2 the ultrasonic vibration can be any ultrasonic vibration of any frequency, for example in frequencies ranging from 18 kHz to 40 MHz.

6. In claim 2 the ultrasonic vibration transducer can be a magnetostriction transducer, a piezoelectric transducer or a capacitive transducer.

7. In claim 2 the attached surgical blade can be any surgical blade on any surgical tool in any shape, style, or material.

8. In claim 7 the surgical blade can be any material that can create incisions or cuts in the tissue.

9. In claim 2 the connection between the ultrasonic vibration transducer and ultrasonic alternating power source can be a wire connection, such a wire or a cable.

10. In claim 2 the connection between the ultrasonic vibration transducer and ultrasonic alternating power source can be a wireless connection, such a pair of antennas for transmitting and receiving, in which the transmitting antenna is connected to the power source and the receiving is connected to the transducer.

11. In claim 2 the cutting of live tissues and blood vessels can be of any tissues and blood vessels on any part of a living body.

12. In claim 11 the living body can be a human body, an animal body, and any other living organism.

13. In claim 2 the ultrasonic alternating power source can be any ultrasonic alternating power source.

14. In claim 2 the ultrasonic vibration can be in direct contact with the tissue or be transmitted over a medium, such as blood, water, and air.

15. In claim 2 the ultrasound waves can be of any amplitude.

16. In claim 2 the bleedingless effect does not have to be complete, which includes any reduction in bleeding.

17. In claim 2 the cutting of tissues can include small incisions to amputations.

18. In claim 2 the bleedingless effect can also be achieved after the cutting has been completed.

19. In claim 1 the effects of the cavitation in the blood vessel, tissue, and/or body fluid include accelerating platelet aggregation and fibrin formation, inducing and enhancing blood clotting.
